(19) United States
(12) Patent Application Publication
(10) Pub. No.: US 2011/0250227 A1
(43) Pub. Date: Oct. 13, 2011

(54) COMPOSITION FOR TREATMENT OF THE SKIN

(76) Inventor: Hanan Elraz, Qiryat Bialick (IL)

(21) Appl. No.: 13/058,760

(22) PCT Filed: Aug. 10, 2009
(86) PCT No.: PCT/IB2009/053504

§ 371 Date: Jul. 4, 2011

Related U.S. Application Data
(60) Provisional application No. 61/136,062, filed on Aug. 11, 2008.

(51) Int. Cl. A61K 36/02; A61K 36/04; A61K 36/03; A61P 31/00; A61K 8/96; A61Q 19/08; A61P 17/00; A61P 29/00; A61K 36/05; A61K 36/45

(52) U.S. Cl. 424/195.17; 424/732

(57) ABSTRACT

A natural composition comprising myrtle berries; seaweed; and at least two of tea tree oil, lemongrass oil, evening primrose oil, ginger, jojoba oil, neem oil, and chamomile, and uses thereof for treatment of skin conditions.

### FIELD OF THE INVENTION

The present invention relates to the field of natural pharmaceuticals, and more specifically to a natural composition comprising myrtle berries; seaweed; and at least two of tea tree oil, lemongrass oil, evening primrose oil, ginger, jojoba oil, neem oil, and chamomile. The present invention further relates to a natural composition comprising myrtle berries, evening primrose oil, neem oil, jojoba oil and almond oil.

### BACKGROUND OF THE INVENTION

The skin is the primary interface between the body and the environment and plays an essential protective role, shielding the body from injury, pathogenic agents, the sun, extremes of temperature, and other environmental factors. In addition, the skin provides thermoregulation, serves as a permeability barrier, and functions as a sensory organ for touch, pressure, pain, itch, and temperature.

Skin is composed of two major layers: the epidermis and the underlying dermis, which are distinct in terms of their architecture, physiology, and function. The epidermis is a stratified epithelium composed of four layers: the stratum basale, stratum spinosum, stratum granulosum, and the outermost stratum corneum. The stratum basale contains a single layer of cuboidal keratinocytes attached to a basement membrane. Above this layer is the spinous layer, characterized by presence of numerous desmosomes.

The stratum granulosum overlies the stratum spinosum and consists of keratinocytes that contain basophilic granules of keratohyalin as well as lamellar granules in the intercellular compartment. The stratum corneum is the most superficial layer and is composed of anucleated, flattened, fully keratinized cells (corneocytes) fused together to form a plate-like structure. The intercellular space is occupied by ordered lipid lamellae that contain specialized proteins and lipids, such as ceramides, fatty acids, and cholesterols, which are secreted from lamellar bodies in the stratum granulosum. The resulting “bricks and mortar” structure provides the stratum corneum with the ability to perform its protective and moisture retaining functions. The thickness of the epidermis ranges from about 75 to 150 μm except on the soles and palms, where it is about 0.4 to 0.6 mm. The dermoepidermal junction (DEJ) is an undulating basement membrane composed primarily of collagen that separates the epidermis from the dermis.

The dermis is a dense, fibroelastic connective tissue that lies beneath the epidermis and provides a strong and flexible supporting layer. It is composed of cells {e.g., fibroblasts), ground substance, and a fibrous network containing collagenous and elastic fibers and also contains blood vessels, nerves, hair follicles, smooth muscle, glands and lymphatic tissue. Collagen, primarily types I, III, V, and VI, forms the majority of the fibrous component, making up about 75% of the dry weight of the dermis and imparting firmness and tensile strength.

Elastin accounts for about 2-4% of dermal proteins and, together with a variety of elastin-associated proteins, forms an interconnecting network of insoluble elastic fibers that are interwoven among the collagen bundles and provide normal skin with its elasticity and resilience. The major component of the mature fiber is a covalently cross-linked polymer of the protein elastin. Elastin is secreted from the cell as a soluble monomer called tropoelastin. In the extracellular space, lysine residues within tropoelastin are specifically modified to form covalent cross-links between tropoelastin chains. This cross-linked polymer has a high degree of reversible distensibility, including the ability to deform to large extensions with small forces.

Many diseases and disorders of the skin are known. These include psoriasis, acne, scabies and hives.

Psoriasis is a common, chronic skin disorder. Plaque psoriasis is the most common type of psoriasis, characterized by red skin covered with silvery scales and inflammation. Patches of circular to oval shaped red plaques that itch or burn are typical of plaque psoriasis. The patches are usually found on the arms, legs, trunk or scalp, but may be found on any part of the skin. The most typical areas are the knees and elbows.

Commonly prescribed drugs for treatment of psoriasis include acitretin, alefacept, amcinonide, ammoniated mercury, anthralin, betamethasone, calcipotriene, clobetasol, coal tar, cortisone, cyclosporine, desomide, desoximetasone, dexamethasone, diflorasone, efalizumab, etanercept, etretinate, fluocinolone, fluocinomide, flurandrenolide, fluticasone, fusidic acid, gentamicin, halconinide, juniper tar, methotrexate, methoxsalen, methylprednisolone, mometasone, prednicarbate, prednisolone, prednisone, salicylic acid, tazarotene, and triamcinolone.

Acne is the most common skin disease in the United States, and results from the action of hormones on the oil glands of the skin, which leads to plugged pores and outbreaks of lesions (pimples). Acne lesions usually occur on the face, neck, back, chest, and shoulders. Although acne is not a serious health threat, severe acne can lead to disfiguring, permanent scarring, which can be upsetting to the affected person.

Treatment options for acne include adapalene, azalaic acid, benzoyl peroxide, clindamycin, dexamethasone, doxycycline, erythromycin, fusidic acid, gentamicin, isotretinoin, minocycline, oxytetracycline, salicylic acid, sodium thiosulfate, sulfacetamide, sulfur, tazarotene, tetracycline and tretinoin.

Scabies is a common infestation of the skin with the microscopic mite Sarcoptes scabei. Scabies spreads rapidly under crowded conditions where there is frequent skin-to-skin contact between people. Commonly prescribed drugs include crotamiton, lindane and permithrin cream.

Urticaria (hives) is an itchy rash consisting of localized swellings of the skin that usually lasts for a few hours before fading away. When urticaria develops around loose tissues of the eyes or lips, the affected area may swell excessively. Commonly prescribed drugs include astemizole, brompheniramine, cetrizine, clemastine, cyprohepatide, desloratidine, diphenydramine, fexofenadine, loratidine, methdilazine, methylprednisolone and triplennamine.

All of the above prescription drugs are associated with at least some side-effects.

Aging and exposure to environmental insults affect the appearance and structure of the skin. Sun-protected, naturally aged skin exhibits epidermal and dermal thinning, fragility, and fine, shallow wrinkles. There is a loss of elastic fibers in the papillary dermis, and collagen fibers become increasingly dense and more randomly oriented. Exposure to ultraviolet radiation from the sun accelerates and alters the degenerative processes associated with aging, resulting in a constellation of changes collectively known as photodamage. As much as 80% of facial aging may well be attributable to sun exposure. Photodamaged skin is characterized by loss of elasticity, deep wrinkles, increased roughness and dryness, and altered pigmentation (age spots). The skin may assume a leathery, thickened appearance characterized by deep furrows.

Various approaches have been developed or are under investigation to reduce the visible signs of photodamage (Stern, R., N. Engl. J. Med, 350:1526-1534, 2004). Use of sunscreens can help to prevent further damage. Topical retinoids (vitamin A derivatives) can reduce the severity of photoaging (U.S. Pat. No. 4,877,805). It has been proposed to treat the skin with compositions containing inhibitors of collagenase or elastase (U.S. Pat. Nos. 5,614,489; 6,884,425). Chemical peels, which involve application of a variety of different chemical compounds to skin, result in temporary improvement in the appearance of photodamaged skin in some individuals. Microdermabrasion and laser resurfacing are other alternatives. Injection of botulinum toxin (e.g., Botox®) has gained popularity as a means of reducing furrows caused by muscle hypertonicity. Skin fillers such as bovine collagen and hyaluronic acid are used for soft tissue augmentation to treat deep wrinkles. However, none of these approaches has provided a satisfactory solution to the problem of damage to skin caused by exposure to the sun. There is a need in the art for improved compositions and methods for caring for photodamaged skin.

There is thus a widely recognized need for, and it would be highly advantageous to have, a composition for treatment of the skin which is devoid of at least some of the limitations of the prior art.

### SUMMARY OF THE INVENTION

The present invention provides compositions comprising myrtle berries; seaweed; and at least two agents selected from the group consisting of tea tree oil, lemongrass oil, evening primrose oil, ginger, jojoba, neem oil, and chamomile.

The present invention further provides a method of treating a skin condition, the method comprising contacting the skin in the area of the condition with a composition comprising myrtle berries; seaweed; and at least two of tea tree oil, lemongrass oil, evening primrose oil, ginger, jojoba oil, neem oil, and chamomile.

According to some embodiments of the composition of the present invention, the two agents comprise tea tree oil and lemongrass oil.

According to some embodiments of the composition of the present invention, the at least two agents comprise evening primrose oil, jojoba oil and neem oil.

The present invention further provides a composition comprising myrtle berries, evening primrose oil, neem oil, jojoba oil, and almond oil.

The present invention further provides a method of treating a skin condition, the method comprising contacting the skin in the area of the condition with a composition comprising myrtle berries, evening primrose oil, neem oil, jojoba oil, and almond oil.

According to some embodiments, the seaweed comprises green algae, red algae, brown algae, or a combination thereof. Optionally and preferably, the seaweed comprises green algae.

According to some embodiments, the concentration of myrtle berries in the composition of the present invention is in the range of from about 10 to about 30% (w/v) of total composition. Optionally and preferably, the concentration of myrtle berries is about 20% (w/v) of total composition.

According to some embodiments, the concentration of green algae in the composition of the present invention is in the range of from about 5 to about 30% (w/v). Optionally and preferably, the concentration of green algae is about 10-15% (w/v) of total composition.

According to some embodiments, the concentration of tea tree oil in the composition of the present invention is in the range of from about 5 to about 20% (v/v) of total composition. Optionally and preferably, the concentration of tea tree oil is about 12-15% (v/v) of total composition.

According to some embodiments, the concentration of lemongrass oil in the composition of the present invention is in the range of from about 5 to about 20% (w/v) of total composition. Optionally and preferably, the concentration of lemongrass oil is about 12% (w/v) of total composition.

According to some embodiments, the concentration of evening primrose oil in the composition of the present invention is in the range of from about 5 to about 30% (w/v) of total composition. Optionally and preferably, the concentration of evening primrose oil is about 15-20% (w/v) of total composition.

According to some embodiments, the concentration of jojoba oil in the composition of the present invention is in the range of from about 15 to about 30% (w/v) of total composition. Optionally and preferably, the concentration of jojoba oil is about 20-25% (w/v) of total composition.

According to some embodiments, the concentration of neem oil in the composition of the present invention is in the range of from about 15 to about 30% (w/v) of total composition. Optionally and preferably, the concentration of neem oil is about 20-25% (w/v) of total composition.

According to some embodiments, the concentration of almond oil in the composition of the present invention is in the range of from about 15 to about 30% (w/v) of total composition. Optionally and preferably, the concentration of almond oil is about 20% (w/v) of total composition.

According to some embodiments, the composition further comprises an emulsifier. The emulsifier may be a surfactant, such as an ionic surfactant (anionic, cationic or zwitterionic) or a non-ionic surfactant.

Examples of suitable anionic surfactants include, without limitation, sodium dodecyl sulfate, ammonium lauryl sulfate, sodium lauryl sulfate and alkyl benzene sulfonate, or combinations thereof.

Examples of suitable cationic surfactants include, without limitation, cetyl trimethylammonium bromide, hexadecyl trimethyl ammonium bromide, cetylpyridinium chloride, polyethoxylated tallow amine, benzalkonium chloride, and benzethonium chloride, or combinations thereof.

Examples of suitable zwitterionic surfactants include, without limitation, dodecyl betaine, dodecyl dimethylamine oxide, cocamidopropyl betaine and cocoamphoglycinate, or combinations thereof.

The non-ionic surfactant may comprise, for example, alkyl poly(ethylene oxide), a copolymer of poly(ethylene oxide) and poly(propylene oxide), an alkyl polyglucoside, a fatty alcohol, and a polysorbate (such as polysorbate 20 or polysorbate 80), or combinations thereof.

The compositions and methods of the present invention are useful for treating a wide range of skin conditions, including, but not limited to, psoriasis, eczema, urticaria, scabies, acne, alopecia areata, bullous pemphigoid, dermatitis, impetigo, keloids, pruritis, rosacea, vitiligo, burns, warts, birthmarks, rashes, pigmentation marks, irritation, inflammation, fungal infection, open wounds, dry skin, photodamage and visible signs of aging.

The present invention further provides a method of treating a skin condition, comprising contacting the skin in the area of the condition with a composition comprising myrtle berries; seaweed; and at least two agents selected from the group consisting of tea tree oil, lemongrass oil, evening primrose oil, ginger, jojoba oil, neem oil, and chamomile.

The present invention further provides a method of treating a skin condition, comprising contacting the skin in the area of the condition with a composition comprising myrtle berries, evening primrose oil, neem oil, jojoba oil, and almond oil.

According to preferred embodiments, “treating” may optionally comprise one or more of preventing, ameliorating, reducing, alleviating, eliminating (partially or completely) or relieving.

The term “contacting” is intended to encompass any manner of locally providing, delivering, or making available to the skin, e.g., to the dermis.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Where ranges are given, endpoints are included within the range. Furthermore, it is to be understood that unless otherwise indicated or otherwise evident from the context and understanding of one of ordinary skill in the art, values that are expressed as a range can assume any specific value or subrange within the stated range in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a photograph of a patient suffering from psoriasis in the region of the elbow, before treatment with the composition of the present invention (FIG. 1a) and 10 days after treatment (FIG. 1b);

FIG. 2 is a photograph of a patient suffering from facial psoriasis, before treatment with the composition of the present invention (FIGS. 2a) and 10 days after treatment (FIG. 2b); and

FIG. 3 is a photograph of a patient suffering from psoriasis of the hands, before treatment with the composition of the present invention (FIGS. 3a) and 10 days after treatment (FIG. 3b).

### DESCRIPTION OF THE PREFERRED EMBODIMENTS

The present invention is of natural compositions for treatment of the skin.

According to some embodiments, the composition comprises myrtle berries; seaweed; and at least two agents selected from the group consisting of tea tree oil, lemongrass oil, evening primrose oil, ginger, jojoba oil, neem oil, and chamomile.

According to some embodiments, the composition comprises myrtle berries, evening primrose oil, neem oil, jojoba oil and almond oil.

Myrtle is an evergreen shrub which produces a round, reddish blue to violet berry. The berries contain the major volatile components α-pinene,1,8-cineole and limonene. The plant has traditionally been used in many herbal remedies including as an astringent, as an antiseptic, for treatment of wounds, and as a decongestant.

Seaweed is a term commonly used for the multicellular marine algae. These include green algae (chlorphyta), red algae (rhodophyta), and brown algae (phaeophyta). Seaweeds are used as sources of food, as fertilizers, and in cosmetics.

Seaweeds are harvested for the extraction of hydrocolloids or phycocolloids, such as alginate, agar and carrageenan. Hydrocolloids have attained commercial significance, especially in food production as food additives. The food industry exploits the gelling, water-retention, emulsifying and other physical properties of these hydrocolloids. Agar is used in foods such as confectionery, meats and poultry products, desserts and beverages and moulded foods. Carrageenan is used in preparation of salad dressings and sauces, dietetic foods, and as a preservative in meat and fish products, dairy items and baked goods. Alginates have many of the same uses as carrageenan, but are also used in production of industrial products such as paper coatings, adhesives, dyes, gels, explosives and in processes such as paper sizing,

In the biomedicinal and pharmaceutical industries, alginates are used in wound dressings, and production of dental moulds and have a host of other applications. In microbiology research, agar is extensively used as culture medium. Seaweed is also a known source of iodine.

Tea tree oil is an essential oil obtained by steam distillation of the leaves of Melaleuca alternifolia, a plant native to Australia. Tea tree oil contains terpenoids, which have been found to have antiseptic and antifungal activity. The compound terpinen-4-ol is the most abundant and is thought to be responsible for most of tea tree oil's antimicrobial activity. Tea tree oil has been use for treatment of acne, athlete's foot, dandruff, vaginitis, thrush, periodontal disease, as an antiseptic, for treatment of boils, eczma, psoriasis, and yeast infections.

Lemongrass oil is extracted from Cymbopogon citratus, a perennial, fast-growing aromatic grass, by steam distillation. The main chemical components of lemongrass oil are myrcene, citronellal, geranyl acetate, nerol, geraniol, neral and traces of limonene and citral. Lemongrass oil is considered to have antimicrobial, antifungal, analgesic, antidepressant and antiseptic properties.

Evening primrose is a plant having yellow flowers that bloom in the evening. Evening primrose oil contains the essential fatty acid, gamma-linolenic acid. Evening primrose oil has been used for treatment of eczema; for treatment of conditions involving inflammation, such as rheumatoid arthritis; for conditions affecting women's health, such as breast pain associated with the menstrual cycle, menopausal symptoms, and premenstrual syndrome; and for cancer and diabetes.

Ginger is a perennial plant containing sesquiterpenoids, with (−)-zingiberene as the main component. Lesser amounts of other sesquiterpenoids (β-sesquiphellandrene, bisabolene and farnesene) and a small monoterpenoid fraction (β-phelladrene, cineol, and citral) have also been identified.

Camomile is an annual, scented herb of the daisy family. The flowers of chamomile provide volatile oils containing alpha-bisabolol, alpha-bisabolol oxides A and B, and matricin. Other active constituents include the bioflavonoids apigenin, luteolin, and quercetin. These active ingredients contribute to chamomile's anti-inflammatory, antispasmodic, and smooth muscle-relaxing effects, particularly in the gastrointestinal tract. It is used externally to spur wound healing and treat inflammation, and internally for fever, digestive upsets, anxiety, and insomnia.

Neem is a fast-growing, evergreen tree of the mahogany family, growing in tropical and semi-tropical regions. Neem oil is used for preparing cosmetics (such as soap, shampoo, balms and creams), and is useful for skin care such as acne treatment, and keeping skin elasticity. Neem oil has been found to be an effective mosquito repellent.

Jojoba oil is a liquid mixture of wax esters produced in the seed of the jojoba plant. Jojoba oil is used as an ingredient in cosmetics and personal care products, especially skin care and hair care, and as a fungicide.

Almond oil is obtained from the dried kernel of the almond plant, and is used as an emollient in many natural skin care products.

The present inventors have surprisingly found that a composition comprising myrtle berries; algae; at least two agents selected from the group consisting of tea tree oil, lemongrass oil, evening primrose oil, ginger, jojoba, neem oil, and chamomile as active ingredients is particularly effective in the treatment of a wide range of skin conditions. The composition of the present invention comprises solely natural ingredients, and is non-toxic, with no side-effects.

According to some embodiments, the composition comprises myrtle berries, green algae, tea tree oil, and lemongrass oil as active ingredients.

According to some embodiments, the composition comprises myrtle berries, green algae, jojoba oil, evening primrose oil and neem oil as active ingredients.

The present inventors have further found that a composition comprising myrtle berries, evening primrose oil, neem oil, jojoba oil and almond oil is surprisingly effective in the treatment of skin conditions such as psoriasis.

According to some embodiments, the concentration of myrtle fruit in the concentration is in the range of from about 10 to about 30% (w/v). Preferably, the concentration is about 20% (w/v).

According to some embodiments, the concentration of green algae in the composition is in the range of from about 5 to about 30% (w/v). Preferably, the concentration is about 10-15% (w/v).

According to some embodiments, the concentration of tea tree oil in the composition is in the range of from about 5 to about 20% (v/v). Preferably, the concentration is about 12-15% (v/v).

According to some embodiments, the concentration of lemongrass oil in the composition is in the range of from about 5 to about 20% (v/v). Preferably, the concentration is about 12% (v/v).

According to some embodiments, the concentration of evening primrose oil in the composition is in the range of from about 5 to about 30% (v/v). Preferably, the concentration is about 15-20% (v/v).

According to some embodiments, the concentration of jojoba oil in the composition is in the range of from about 15 to about 30% (v/v). Preferably, the concentration is about 20-25% (v/v).

According to some embodiments, the concentration of neem oil in the composition is in the range of from about 15 to about 30% (v/v). Preferably, the concentration is about 20-25% (v/v).

According to some embodiments, the concentration of almond oil in the composition is in the range of from about 15 to about 30% (v/v). Preferably, the concentration is about 20% (v/v).

The composition preferably further comprises an emulsifier and a diluent.

The emulsifier may be any surfactant, including an ionic (anionic, cationic or zwitterionic), or nonionic surfactant.

Non-limiting examples of suitable anionic surfactants include, for example, sodium dodecyl sulfate, ammonium lauryl sulfate, sodium lauryl sulfate and alkyl benzene sulfonate.

Non-limiting examples of suitable cationic surfactants include, for example, cetyl trimethylammonium bromide, hexadecyl trimethyl ammonium bromide, cetylpyridinium chloride, polyethoxylated tallow amine, benzalkonium chloride, and benzethonium chloride.

Non-limiting examples of suitable zwitterionic surfactants include, for example, dodecyl betaine, dodecyl dimethylamine oxide, cocamidopropyl betaine and cocoamphoglycinate.

The non-ionic surfactant may comprise, for example, one or more of alkyl poly(ethylene oxide), a copolymer of poly(ethylene oxide) and poly(propylene oxide), an alkyl polyglucoside (such as octyl glucoside or decyl maltoside), a fatty alcohol (such as cetyl alcohol or oleyl alcohol), and a polysorbate (such as Tween 20 or Tween 80).

Optionally and preferably, the emulsifier comprises Tween 20.

The diluent is preferably sterilized water. The water is sterilized at temperature of greater than 40° C.

According to some embodiments, the composition of the present invention may optionally further comprise one or more additional ingredients. The additional ingredient may be an active ingredient and/or an excipient.

Examples of suitable additional active ingredients include, for example, vitamins, proteins, amino acids, small molecules, drugs, plant extracts, sunscreen agents, etc.

Exemplary vitamins include vitamin A, vitamin Bi (thiamine), vitamin B₂ (riboflavin), vitamin B₃ (niacin), vitamin B compounds, retinoids, panthenol, niacinamide, retinol, retinyl propionate, retinyl palmitate, retinoic acid, flavonoids (chalcones, chromones, flavones, isoflavones), vitamin B₄ (adenine), vitamin B5 (pantothenic acid), vitamin B₆ (pyridoxine), vitamin B₇ (biotin), vitamin B9 (folic acid), vitamin B12 (cyanocobalamin), vitamin C (ascorbic acid), vitamin D (ergocalciferol), vitamin E (tocopherol), tocopherol acetate, vitamin K, and combinations thereof.

A protein, peptide, or amino acid may optionally be added to the inventive composition to nourish the skin, impart a desired characteristic to skin, or impart a desired characteristic to the composition (e.g., thickening the composition).

Exemplary proteins include elastin, fibrillin, fibronectin, laminin, keratin, proteoglycans, wheat protein, gelatin, collagen, silk, soy protein, wheat protein, rice protein, corn protein, jojoba protein, milk protein, whey protein, casein, albumin, egg protein, and fragments thereof. As would be appreciated by one of ordinary skill in the art, derivatives, mutants, fusion proteins, fragments, or combinations of any of these proteins may also be included in the inventive cosmetic composition.

Any of the twenty natural amino acids may be included in the inventive composition. In certain embodiments, the amino acid used in the inventive compostion is selected from the group consisting of phenylalanine, valine, tryptophan, threonine, isoleucine, methionine, cysteine, homocysteine, histidine, arginine, lysine, leucine, proline, serine, aspartate, glutamate, glutamine, and combinations thereof.

Plant extract may optionally be added to the inventive composition to nourish the skin, provide a fragrance or color to the composition, impart a desired characteristic to the treated skin, or impart a desired characteristic to the composition.

Exemplary plants that can be used to prepare plant extracts include, but are not limited to, birch, rosemary, arnica, hamamelis, sage, St. John's bread, henna, hop, lime, orange, lemon, grapefruit, aloe, thyme, calendula, horsetail, mountain gentian, nettle, chestnut, avocado, milfoil, coltsfoot, marigold, peach, rose, senna, mint, white lilly, lavender, grape seed, bayberry, saw palmetto, tea tree, soy bean, almond, peanut, sea buckthorn seed, hibiscus, horsetail, rasberry, rose hips, and olive.

The composition may optionally include a sunscreen agent to protect the skin from the damaging ultraviolet rays of the sun, including UV-A and/or UV-B rays. Exemplary sunscreen agents include aminobenzoic acid (PABA), PABA-derivatives (e.g., allantoin PABA, butyl PABA, dimethyl PABA ethyl cetearyldimonium tosylate, ethyl dihydroxypropyl PABA, ethylhexyl dimethyl PABA, N-ethyl-3-nitro PABA, ethyl PABA, glyceryl PABA, PEG-25 PABA, pentyl dimethyl PABA, and triP ABA panthenol), avobenzone, cinoxate, dioxybenzone, homosalate, menthyl anthranilate, octocrylene, octyl methoxycinnamate, octyl salicylate, oxybenzone, padimate O, phenylbenzimidazole sulfonic acid, sulisobenzone, titanium dioxide, trolamine salicylate, zinc oxide, ecamsule, terephthalylidene dicamphor sulfonic acid, 4-methylbenzylidene camphor, bisoctrizole, methylene bis-benzotriazolyl tetramethylbutylphenol, bemotrizinol, bis-ethylhexyloxyphenol methoxyphenyl triazine, drometrizole trisiloxane, bisdisulizole disodium, disodium phenyl dibenzimidazole tetrasulfonate, diethylamino hydroxybenzoyl hexyl benzoate, octyl triazone, ethylhexyl triazone, iscotrizinol, diethylhexyl butamido triazone, polysilicone-15, hydroxyphenylbenzotriazole, isoamyl p-methoxycinnamate, benzophenone-3 (oxybenzone), benzophenone-4 (sulisobenzone), benzophenone-8 (dioxybenzone), butyl methoxydibenzoylmethane (avobenzone), cinoxate, DEA-methoxycinnamate, digalloyl trioleate, dimethicone/PEG-15 crosspolymer, 1-(3,4-dimethoxyphenyl)-4,4-dimethyl-1,3-pentanediene, ethylhexyl methoxycinnamate (octinoxate), ethylhexyl salicylate (octisalate), 4-(2-beta-glucopyranosiloxy) propoxy-2- hydroxybenzophenone, Helianthus annuus (sunflower) seed extract, homosalate, hydrolyzed linseed extract, lawsone, menthyl anthranilate (meradimate), octocrylene, phenylbenzimidazole sulfonic acid (ensulizole), Pinus pinaster bark extract, red petrolatum, Spirulina platensis powder, TEA-salicylate (trolamine salicylate), titanium dioxide, Vitis vinifera (grape) seed extract,and combinations thereof.

According to some embodiments, the composition of the present invention may optionally include one or more excipients, such as preservatives, antioxidants, and/or chelating agents to extend the shelf-life and/or prevent the degradation of the components of the composition.

Suitable antioxidants include reducing agents and/or free radical scavengers.

Exemplary preservative, antioxidants, and chelating agents include vitamin C (ascorbic acid), glutathione, lipoic acid, uric acid, carotenes {e.g., beta-carotene), alpha-tocopherol (vitamin E), ubiquinol (coenzyme Q), melatonin, ethylenediamine tetraacetic acid (EDTA) and salts thereof, citric acid and salts thereof, EGTA, aminotrimethylene phosphonic acid, resveratrol, flavonoids, lycophene, propyl gallate, tertiary butylhydroquinone, butylated hydroxyanisole, butylated hydroxytoluene, benzoates, nitrites, nitrates, sulfites, calcium propionate, sodium bisulfite, potassium hydrogen sulfite, benzyl alcohol, methyl paraben, propyl paraben, imidazolidinylurea, sulfur dioxide, and combinations thereof.

According to some embodiments, the composition may optionally comprise an emollient. Suitable emollient materials include branched chain hydrocarbons having an average molecular weight of from about 100 to about 15,000, methyl isostearate, isopropyl isostearate, isostearyl neopentanoate, isononyl isononanoate, isodecyl octanoate, isodecyl isononanoate, tridecyl isononanoate, myristyl octanoate, octyl pelargonate, octyl isononanoate, myristyl myristate, myristyl neopentanoate, myristyl octanoate, myristyl propionate, isopropyl myristate and mixtures thereof.

According to some embodiments, the composition of the present invention may optionally include a humectant.

According to some embodiments, the composition of the present invention may optionally contain a thickening agent.

According to some embodiments, the composition of the present invention may optionally comprise a penetration enhancer. As used herein, a “penetration enhancer” is an agent or combination of agents that increases delivery of a second agent across the SC or a portion thereof, such that an increased amount of the second agent reaches the dermis relative to the amount of the second agent that would reach the dermis in the absence of the penetration enhancer under otherwise identical conditions.

Exemplary penetration enhancers include various oils, fatty acids, lipases, alcohols, surfactants, etc.

Examples of pentration enhancers include benzyloxy ethanol, benzyl alcohol, phenoxy ethanol, phenoxy isopropanol, methyl phenoxy ethanol, benzyl glycerol, N-benzyl formide, N-methylpyrrolidone, N-ethyl pyrrolidone, cinnamyl alcohol, phenethyl alcohol, p-methyl benzyl alcohol, butyl cellosolve, methyl carbitol, ethyl carbitol, propyl carbitol, butyl carbitol, diethyleneglycol, diethyl ether, and dipropyleneglycol diethyl ether.

According to some embodiments, the composition of the present invention may optionally include a fragrance or perfume.

According to some embodiments, the composition of the present invention may be used for treatment of a wide range of skin conditions, including, for example, psoriasis, eczema, urticaria (hives), scabies, acne, alopecia areata, bullous pemphigoid, dermatitis, impetigo, keloids, pruritis, rosacea, vitiligo, burns (including sunburn), warts, birthmarks, rashes, pigmentation marks, irritation, inflammation, fungal infection (such as athlete's foot, fungal infection of the nail bed), open wounds, dry skin, photodamage, and visible signs of aging.

The present invention further provides a method of treating a skin condition, the method comprising contacting the skin in the area of the condition with a composition comprising myrtle berries; seaweed; and at least two agents selected from the group consisting of tea tree oil, lemongrass oil, evening primrose oil, ginger, and camomile.

“Photodamage” refers to a variety of alterations in the appearance and structure of the skin that occur as a consequence of exposure to the sun, and which may be particularly severe if the skin has been experienced significant and extended exposure. These alterations include wrinkles (e.g., folds, furrows, or creases in the skin), alterations and irregularities in pigmentation (e.g., age spots, sallowness), pebbly texture and appearance, increased roughness and dryness, reduced skin tone and elasticity, accumulation of elastotic material, loss of collagen, etc. It will be appreciated that not all of these changes need be present in order for an individual's skin to be considered photodamaged. The severity of photodamage will vary depending on a variety of factors such as amount of sun exposure, age, genetic makeup, health, and habits of the individual, exposure to other environmental conditions such as dry air, pollution, cigarette smoke, etc.

Visible signs of aging include, but are not limited to, all outward visible and/or tactilely perceptible manifestations, as well as any other macroscopic or microscopic changes attributable at least in part to aging of skin. These signs include, but are not limited to, the development of textural discontinuities such as wrinkles (e.g., fine wrinkles and/or coarse deep wrinkles), furrows, creases, skin lines, crevices, bumps, large pores (e.g., associated with adnexal structures such as sweat gland ducts, sebaceous glands, or hair follicles), unevenness or roughness, loss of skin elasticity, sagging, loss of skin firmness, loss of skin tightness, loss of skin recoil from deformation, discoloration, blotching, sallowness, hyperpigmented skin regions such as age spots and freckles, keratoses, telangiectasia, spider vessels, etc.

According to some embodiments, the composition is applied topically.

It has further been found that in addition to the local effect produced at the site of application, the composition of the present invention also produces a beneficial effect on skin conditions at other locations. A portion of the topically applied composition enters the circulatory system by transdermal absorption, and is transported to other sites of action.

The compositions of the invention can be provided in any form that delivers an effective amount of the active agents to the skin. Suitable forms include, but are not limited to, soaps, wipes, gels, hydrogels, creams, ointments, lotions, pastes, suspensions, emulsions, sprays, patches (e.g., hydrogel patch), masks, and powders.

The inventive compositions and methods can be employed at various time intervals and over different periods of time as desired to achieve the desired result, e.g., multiple times per day (e.g., in the morning and evening), daily, every other day, once a week, once a month, bimonthly, etc. for between about 1 to 10 weeks or longer, e.g., indefinitely. The skilled artisan will appreciate that certain factors can influence the amount and timing required to provide a the desired improvement in the skin, including but not limited to the severity of the condition, previous and concurrent treatments, age of the subject, etc. Generally, treatment of a subject with an inventive composition can include a single treatment or, in many cases, can include a series of treatments.

As described in detail in the Examples section below, an exemplary composition was tested for hypollergenic reactions, on 50 volunteer subjects. The test showed a 100% success rate.

The duration of treatment depends upon the particular condition being treated. For example, an improvement in the skin of psoriasis patients is seen within about 5 weeks of treatment.

As described in detail in the Examples section below, the composition of the present invention is tested on a large number of subjects, including those suffering from psoriasis, acne and sunburn.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Example 1

### Representative Composition I

An exemplary liquid composition in accordance with the principles of the present invention comprises, per 100 ml:

| | |
|---|---|
| myrtle berries | 20 g |
| lemongrass | 12 ml |
| green algae | 10 g |
| tea tree oil | 8 ml |
| Tween 20 | 5 ml |
| water | 75 ml |

### Example 2

### Preparation of the Composition

A composition according to Example 1 was prepared as follows.

Myrtle fruit was picked in Israel in December when ripe, and of a purple color. The fruit was cleaned by steam treatment, and an incision made in each fruit using a sterile surgical blade. The fruit was then placed in a glass container filled with Australian tea tree oil at room temperature.

Green algae were collected from Achziv Beach, northern Israel, in the summer months and subjected to steam cleaning. The algae were then sun-dried for about one week. The dried algae were then finely shredded and added to the container of tea tree oil and myrtle fruit. The mixture was allowed to stand for about 5 weeks.

The tea tree oil provides acidic and anti-bacterial agents which causes the myrtle fruit and the algae to release various vitamins and other agents which have a beneficial effect a range of skin conditions.

The mixture was then concentrated and transferred to a second container, containing a low concentration of Tween 20, and mixed for about one hour. Sterilized water was then added at a temperature of about 40° C., and stirred for about 3 hours. The composition was allowed to stand for at least 2 days prior to use.

### Example 3

### Representative Composition II

20% myrtle berries

15% green algae

15% evening primrose oil

25% jojoba oil

25% neem oil

### Example 4

### Representative Composition III

20% myrtle berries

20% evening primrose oil

20% neem oil

20% jojoba oil

20% almond oil

### Example 5

### Pilot Study on the Effect of Composition I on Treatment of Psoriasis

Tests are carried out on a sample group of about 5 subjects diagnosed as suffering from a high level of psoriasis vulgaris. An affected area, such as the leg, is selected and photographed. The composition of the present invention, in accordance with Examples 1 and 2, are applied to the affected area. The area is photographed at 24 hours and 48 hours after application, and at 5 and 10 days after application. The extent of psoriasis is assessed by inspection of the photographs, and comparison with the photograph obtained prior to application.

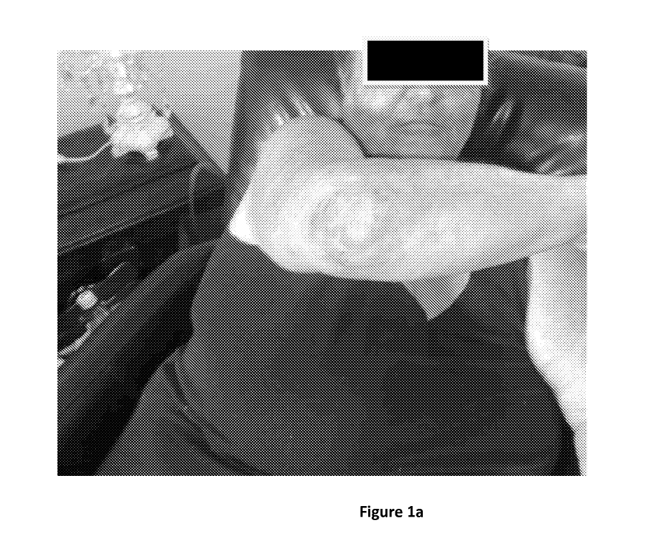

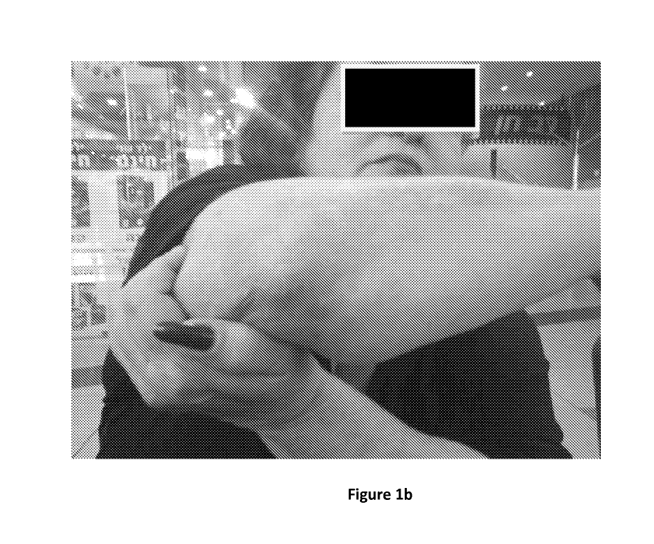

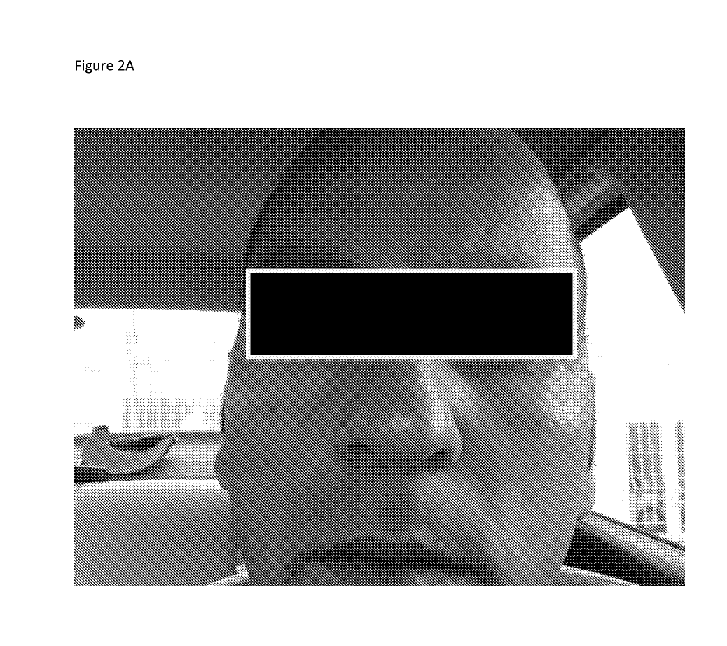

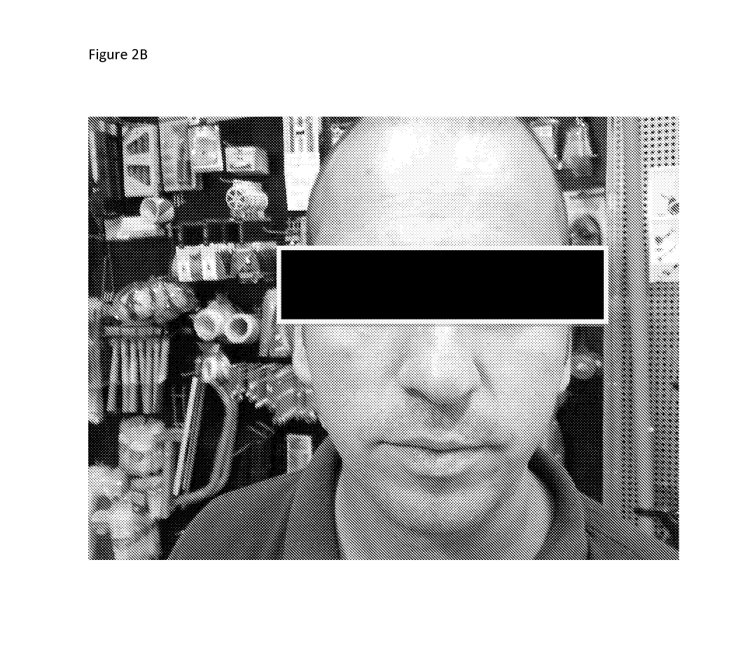

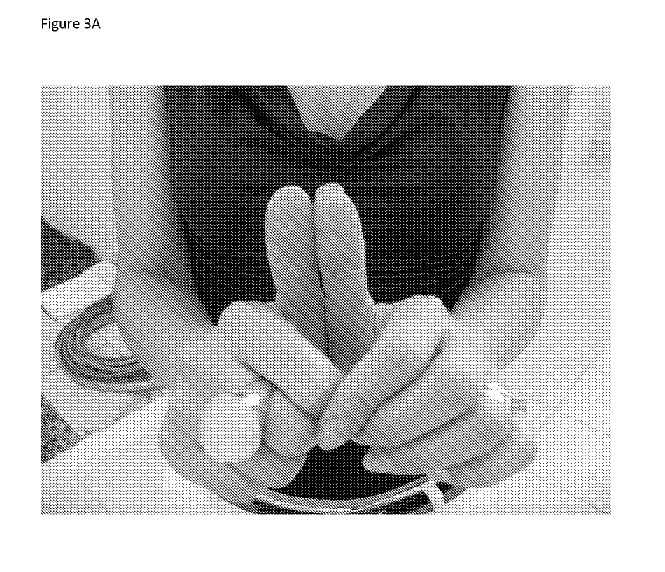

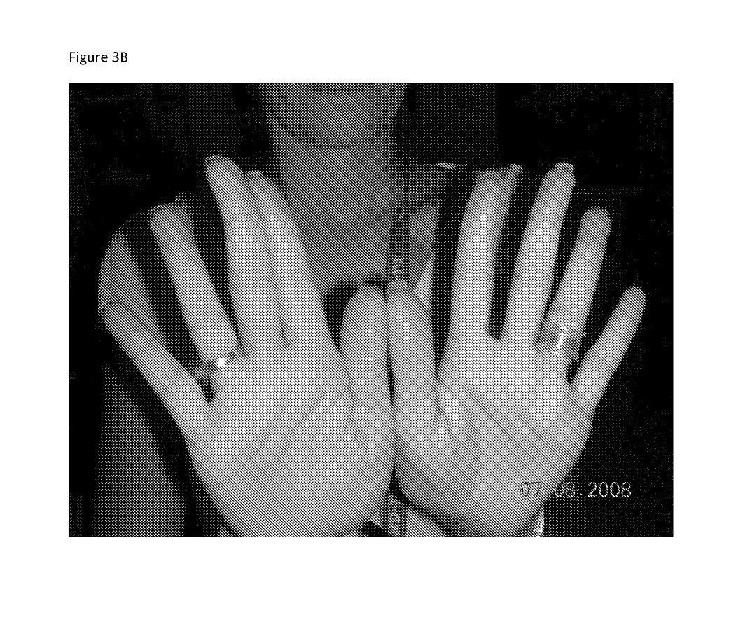

Photographs of a patient taken before and after treatment with composition I, are shown in FIGS. 1a and 1b, respectively.

### Example 6

### Further Pilot Study on the Effect of Composition I on Treatment of Psoriasis

Ten subjects between the ages of 18 and 65 are studied. All subjects are suffering from light to medium levels of psoriasis.

The test composition is applied topically twice per day (morning and evening) for a period of 4 weeks. Subjects are examined prior to commencement of treatment, and after 2 and 4 weeks of treatment, at the Hila Skin Research Institute, Israel. Levels of skin redness, pigmentation are assessed by visual inspection, according to a defined scale; the treatment area is photographed; and the degree of redness measured using a Derma Spectrometer (Cortex Technology) or similar spectrometer. Additionally, at each visit to the Institute, lesions are evaluated in terms of plaque thickness, scaling, and area of plaque, by a qualified assessor.

Furthermore, each subject is required to complete a questionnaire upon completion of the treatment, regarding his/her impression of the effectiveness of the treatment in terms of parameters such as decreased itching etc; and regarding satisfaction with the composition as expressed by the intention to purchase further quantities of the composition, and comparison with other compositions which may have been used previously by the subject.

### Example 7

### Effect of Representative Compositions on Treatment of Eczema

For each composition I-III, tests are carried out on a sample group of about 5 subjects diagnosed as suffering from a high level of eczema. An affected area, such as the leg, is selected and photographed. The compositions of the present invention, in accordance with Examples 1 to 4, are applied to the affected area. The area is photographed at 24 hours and 48 hours after application, and at 5 and 10 days after application. The extent of exzema is assessed by inspection of the photographs, and comparison with the photograph obtained prior to application.

### Example 8

### Effect of Representative Compositions on Treatment of Acne

For each composition I-III, tests are carried out on a sample group of about 5 subjects diagnosed as suffering from acne. An affected area of the face is selected and photographed. The compositions of the present invention, in accordance with Examples 1 to 4, are applied to the affected area. The area is photographed at 24 hours and 48 hours after application, and at 5 and 10 days after application. The extent of acne is assessed by inspection of the photographs, and comparison with the photograph obtained prior to application.

### Example 9

### Effect of Composition I on Treatment of Xcabies

The composition I of the present invention in liquid form, in accordance with Examples 1 and 2, was applied to the skin of 81 volunteer subjects suffering from scabies. Within about 5 days, all patients were found to be free of signs of scabies.

### Example 10

### Hypoallergenic Testing of Composition I

### Method

The aim of the study was to determine the sensitizing properties of the test article using Draize Repeated Insult Patch Test in human volunteers.

Hypoallergenic testing was carried out at the Institute for Skin Research (ISR), Tel Aviv, on 50 volunteer subjects, 5 males and 45 females, ranging in age from 18 to 65 years. 12 subjects were aged 18 to 35 years; 19 subjects were aged 36 to 45 years; and 19 subjects were aged 46 to 65 years.

The study was carried out in accordance with the Good Clinical Practice and Standards established by the International Standards Organization (ISO), and the standard operating procedure of the ISR.

The characteristics of the subjects are summarized in Table 1:

TABLE 1

| | | MEDICAL HISTORY LIKELY |
|---|---|---|
| SEX | AVERAGE AGE | TO INFLUENCE THE STUDY |
| F | 43.3 | none |
| M | 42.6 | none |

Exclusion criteria comprised pregnant or nursing women; cutaneous pathology on the treated zone; subjects suffering from serous or progressive diseases; subjects using a treatment (retinoids, anti-inflammatory substances such as steroids) and modifiers of the cutaneous hydration; unstable weight; and excessive use of alcohol or tobacco.

The sensitizing properties were evaluated by a patch preparation consisting of an occlusive application of the product by Finn Chamber/Hill Top Chamber/Leukotest, or any other similar chamber on the volunteers. Each patch contained the test material.

The method employed in carrying out the test was similar to that described in “Appraisal of the Safety of Chemicals in Foods, Drugs and Cosmetics” by J. H. Draize, and published by the Association of Food and Drug Officials of the United States.

The test involved the application of the test article on the intrascapular region of the back, or the arm of a group of 50 volunteer subjects. Subjects were determined to be in good general health and free of any visible skin disease or anomaly in the area to be patched. Each subject was required to read, understand and sign an informed consent statement.

The test article was either tested as supplied, or diluted.

Soaps were diluted 10-20 fold in water. For example, for a 20 fold dilution, 2 g of the material and 38 g of water (total weight 40 g) were stirred with a magnetic rod or on a stiffing plate until a homogenous solution was obtained The final concentration of the product was 5% (w/w). 0.07-1.0 ml or g of the test material was applied in a patch test.

For test of wipes, the test was conducted on a mixture of the wipe fabric and the material with which the wipe was impregnated.

The patch was applied to its designated contact site and remained in place for 24 hours. At the end of this period, the patch was removed and the site was examined for any dermal response. The subjects rested for 24 hours, after which the skin site was examined again. A patch was then applied to the same site as previously used. The second application was identical to the first and remained in place for 24 hours. This procedure was repeated on alternate days until a series of nine applications were made. The subjects examined the site for any dermal response and reported their observations prior to the next application. The same site was used throughout the study. Each application was applied and removed by the staff of the Institute. Adherence to the study protocol was monitored.

No water was applied to the test site during application of the patch. No systemic or topical treatment likely to modify the skin was permissible. No use of dermopharmaceutical or cosmetic products, including cleansing products, was permissible on the zones being evaluated.

Following the ninth application, a rest period of two weeks elapsed, after which a challenge application was applied in the same manner and to the same site as described above. The challenge application was removed after 24 hours and the site was examined and graded for signs of irritation or sensitization. A follow-up examination was conducted at 48 hours after the challenge application (24 hours after patch removal) as well as at 48 hours and 72 hours after patch removal.

A grading scale was used as follows:
0 no visible reaction
? doubtful reaction; faint, minimal erythema, no infiltration
1 weak positive reaction: erythema, infiltration, discrete papules
2 strong positive reaction: erythema, infiltration, papules, discrete vesicles
3 extra positive reaction: intense erythema, infiltration, coalescing vesicles/bullous reaction
IR irritation reaction: discrete erythema without infiltration/patchy follicular erythema/hemorrhage and follicular pustules
NT not tested

An adverse event was defined as any clinical or biological alteration to the initial condition of the subject (including intercurrent diseases), regardless of whether it was related to the test product. Serious adverse events include hospitalization, life-threatening conditions, death, and sequellae or partial invalidism. Conditions for exit from the test were in accordance with the Helsinki/Tokyo/Venice declaration and Israeli law concerning the protection of subjects in biomedical research. Subjects had the right to leave the study at any time and for any reason. The investigator was authorized to terminate observation at any time if there was an intercurrent disease or any untoward event. The company could demand a subject be excluded from the test for violation of the protocol, for administrative reasons, or for any reason whatsoever.

### Results

### Pilot Study of Composition I: Subject Questionnaire

i. Patient aged 44, medium level of psoriasis on lower right limb, reported an improvement of skin condition and decrease in skin lesions;
ii. Patient having medium level of psoriasis on T panel of face reported an improvement in skin condition after the first application of the composition;
iii. Patient aged 34, medium level of psoriasis on palms of hands, reported an improvement in skin condition after 2 applications of the composition, resulting in smooth skin following continued use;
iv. Patient aged 39, severe level of psoriasis on left elbow including itching and peeling of skin, reported an improvement upon use of the composition, disappearance of itching and improved appearance of skin, resulting in almost complete disappearance of signs of psoriasis.

### Hypoallergenic Testing of Composition I

All subjects completed the study.

Results are shown in Tables 2 and 3.

TABLE 2

| | | | | | RESULTS ACCORDING TO THE GRADING SCALE | | | | | | | | | |
|---|---|---|---|---|---|---|---|---|---|---|---|---|---|---|
| NO | SUBJECT | PRODUCT | SEX | AGE | 1 | 2 | 3 | 4 | 5 | 6 | 7 | 8 | 9 | 10 |
| 1 | C. T. | 1 | F | 51 | 0 | 0 | 0 | 0 | 0 | 0 | 0 | 0 | 0 | 0 |
| 2 | K. S. | 1 | F | [symbol] 2 | 0 | 0 | 0 | 0 | 0 | 0 | 0 | 0 | 0 | 0 |
| 3 | A. [symbol] | 1 | F | [symbol] 0 | 0 | 0 | 0 | 0 | 0 | 0 | 0 | 0 | 0 | 0 |
| 4 | S. L. | 1 | F | 35 | 0 | 0 | 0 | 0 | 0 | 0 | 0 | 0 | 0 | 0 |
| 5 | N. N. | 1 | F | 44 | 0 | 0 | 0 | 0 | 0 | 0 | 0 | 0 | 0 | 0 |
| 6 | B. S. | 1 | F | 36 | 0 | 0 | 0 | 0 | 0 | 0 | 0 | 0 | 0 | 0 |
| 7 | K. S. | 1 | F | 50 | 0 | 0 | 0 | 0 | 0 | 0 | 0 | 0 | 0 | 0 |
| 8 | K. M. | 1 | F | 46 | 0 | 0 | 0 | 0 | 0 | 0 | 0 | 0 | 0 | 0 |
| 9 | L. K. | 1 | F | [symbol] | 0 | 0 | 0 | 0 | 0 | 0 | 0 | 0 | 0 | 0 |
| 10 | K. [symbol] | 1 | F | 37 | 0 | 0 | 0 | 0 | 0 | 0 | 0 | 0 | 0 | 0 |
| 11 | S. H. | 1 | F | 30 | 0 | 0 | 0 | 0 | 0 | 0 | 0 | 0 | 0 | 0 |
| 12 | C. [symbol] | 1 | F | 35 | 0 | 0 | 0 | 0 | 0 | 0 | 0 | 0 | 0 | 0 |
| 13 | I. [symbol] | 1 | M | 45 | 0 | 0 | 0 | 0 | 0 | 0 | 0 | 0 | 0 | 0 |
| 14 | I. V. | 1 | F | 40 | 0 | 0 | 0 | 0 | 0 | 0 | 0 | 0 | 0 | 0 |
| 15 | B. S. | 1 | F | 40 | 0 | 0 | 0 | 0 | 0 | 0 | 0 | 0 | 0 | 0 |
| 16 | [symbol] A. | 1 | F | 50 | 0 | 0 | 0 | 0 | 0 | 0 | 0 | 0 | 0 | 0 |
| 17 | R. O. | 1 | F | 37 | 0 | 0 | 0 | 0 | 0 | 0 | 0 | 0 | 0 | 0 |
| 18 | I. R. | 1 | F | 40 | 0 | 0 | 0 | 0 | 0 | 0 | 0 | 0 | 0 | 0 |
| 19 | [symbol] | 1 | F | 55 | 0 | 0 | 0 | 0 | 0 | 0 | 0 | 0 | 0 | 0 |
| 20 | C. [symbol] | 1 | F | 50 | 0 | 0 | 0 | 0 | 0 | 0 | 0 | 0 | 0 | 0 |
| 21 | A. N. | 1 | F | 44 | 0 | 0 | 0 | 0 | 0 | 0 | 0 | 0 | 0 | 0 |
| 22 | M. L. | 1 | F | 37 | 0 | 0 | 0 | 0 | 0 | 0 | 0 | 0 | 0 | 0 |
| 23 | T. O. | 1 | F | 53 | 0 | 0 | 0 | 0 | 0 | 0 | 0 | 0 | 0 | 0 |
| 24 | G. K. | 1 | F | 47 | 0 | 0 | 0 | 0 | 0 | 0 | 0 | 0 | 0 | 0 |
| 25 | R. M. | 1 | F | 37 | 0 | 0 | 0 | 0 | 0 | 0 | 0 | 0 | 0 | 0 |
| 26 | G. H. | 1 | F | 64 | 0 | 0 | 0 | 0 | 0 | 0 | 0 | 0 | 0 | 0 |
| 27 | G. S. | 1 | F | 38 | 0 | 0 | 0 | 0 | 0 | 0 | 0 | 0 | 0 | 0 |
| 28 | N. I. | 1 | F | 30 | 0 | 0 | 0 | 0 | 0 | 0 | 0 | 0 | 0 | 0 |
| 29 | T. N. | 1 | F | [symbol] | 0 | 0 | 0 | 0 | 0 | 0 | 0 | 0 | 0 | 0 |
| 30 | [symbol] F. | 1 | F | [symbol] | 0 | 0 | 0 | 0 | 0 | 0 | 0 | 0 | 0 | 0 |
| 31 | T. L. | 1 | F | 30 | 0 | 0 | 0 | 0 | 0 | 0 | 0 | 0 | 0 | 0 |
| 32 | A. L. | 1 | M | 65 | 0 | 0 | 0 | 0 | 0 | 0 | 0 | 0 | 0 | 0 |
| 33 | A. Y. | 1 | F | 63 | 0 | 0 | 0 | 0 | 0 | 0 | 0 | 0 | 0 | 0 |
| 34 | R. A. | 1 | F | 30 | 0 | 0 | 0 | 0 | 0 | 0 | 0 | 0 | 0 | 0 |
| 35 | A. S. | 1 | M | 27 | 0 | 0 | 0 | 0 | 0 | 0 | 0 | 0 | 0 | 0 |
| 36 | B. B. | 1 | M | 41 | 0 | 0 | 0 | 0 | 0 | 0 | 0 | 0 | 0 | 0 |
| 37 | A. M. | 1 | F | 35 | 0 | 0 | 0 | 0 | 0 | 0 | 0 | 0 | 0 | 0 |
| 38 | D. N. | 1 | M | 35 | 0 | 0 | 0 | 0 | 0 | 0 | 0 | 0 | 0 | 0 |
| 39 | L. N. | 1 | F | 42 | 0 | 0 | 0 | 0 | 0 | 0 | 0 | 0 | 0 | 0 |
| 40 | S. V. | 1 | F | 35 | 0 | 0 | 0 | 0 | 0 | 0 | 0 | 0 | 0 | 0 |
| 41 | Z. N. | 1 | F | 4 [symbol] | 0 | 0 | 0 | 0 | 0 | 0 | 0 | 0 | 0 | 0 |
| 42 | D. C. | 1 | F | 52 | 0 | 0 | 0 | 0 | 0 | 0 | 0 | 0 | 0 | 0 |
| 43 | S. T. | 1 | F | 50 | 0 | 0 | 0 | 0 | 0 | 0 | 0 | 0 | 0 | 0 |
| 44 | S. M. | 1 | F | 63 | 0 | 0 | 0 | 0 | 0 | 0 | 0 | 0 | 0 | 0 |
| 45 | M. N. | 1 | F | 55 | 0 | 0 | 0 | 0 | 0 | 0 | 0 | 0 | 0 | 0 |
| 46 | [symbol] | 1 | F | 47 | 0 | 0 | 0 | 0 | 0 | 0 | 0 | 0 | 0 | 0 |
| 47 | N. L. | 1 | F | 34 | 0 | 0 | 0 | 0 | 0 | 0 | 0 | 0 | 0 | 0 |
| 48 | [symbol] N. | 1 | F | 47 | 0 | 0 | 0 | 0 | 0 | 0 | 0 | 0 | 0 | 0 |
| 49 | O. A. | 1 | F | 32 | 0 | 0 | 0 | 0 | 0 | 0 | 0 | 0 | 0 | 0 |
| 50 | M. A. | 1 | F | 63 | 0 | 0 | 0 | 0 | 0 | 0 | 0 | 0 | 0 | 0 |

[symbol]

indicates data missing or illegible when filed

TABLE 3

| | | | | 20 MINUTES | 24 HOURS AFTER | 48 HOURS AFTER |
|---|---|---|---|---|---|---|
| | | | | AFTER REMOVAL | REMOVAL OF | REMOVAL OF |
| | | | | OF THE 10^{th} | THE 10^{th} | THE 10^{th} |
| | | | | APPLICATION | APPLICATION | APPLICATION |
| No | Subject | Sex | Age | DATE Jun. 5, 2008 | DATE Jul. 5, 2008 | DATE Aug. 5, 2008 |
| 1 | C. T. | F | 51 | 0 | 0 | 0 |
| 2 | K. S. | F | 32 | 0 | 0 | 0 |
| 3 | A. T. | F | 40 | 0 | 0 | 0 |
| 4 | S. L. | F | 35 | 0 | 0 | 0 |
| 5 | S. S. | F | 44 | 0 | 0 | 0 |
| 6 | D. S. | F | 36 | 0 | 0 | 0 |
| 7 | K. S. | F | 50 | 0 | 0 | 0 |
| 8 | K. M. | F | 4 [symbol] | 0 | 0 | 0 |
| 9 | [symbol] K. | F | 32 | 0 | 0 | 0 |
| 10 | R. [symbol] | F | 37 | 0 | 0 | 0 |
| 11 | S. [symbol] | F | 30 | 0 | 0 | 0 |
| 12 | C. P. | F | [symbol] 5 | 0 | 0 | 0 |
| 13 | [symbol] | M | [symbol] 5 | 0 | 0 | 0 |
| 14 | I. V. | F | 40 | 0 | 0 | 0 |
| 15 | B. S. | F | 40 | 0 | 0 | 0 |
| 16 | [symbol] A. | F | 50 | 0 | 0 | 0 |
| 17 | R. O. | F | 37 | 0 | 0 | 0 |
| 18 | I. R. | F | 40 | 0 | 0 | 0 |
| 19 | H. R. | F | 55 | 0 | 0 | 0 |
| 20 | C. H. | F | 50 | 0 | 0 | 0 |
| 21 | A. N. | F | 44 | 0 | 0 | 0 |
| 22 | M. [symbol] | F | [symbol] 7 | 0 | 0 | 0 |
| 23 | T. O. | F | 53 | 0 | 0 | 0 |
| 24 | [symbol] K. | F | 47 | 0 | 0 | 0 |
| 25 | R. M. | F | 37 | 0 | 0 | 0 |
| 26 | G. H. | F | 64 | 0 | 0 | 0 |
| 27 | G. S. | F | 38 | 0 | 0 | 0 |
| 28 | N. I. | F | 40 | 0 | 0 | 0 |
| 29 | T. N. | F | 3 [symbol] | 0 | 0 | 0 |
| 30 | B. [symbol] | F | 38 | 0 | 0 | 0 |
| 31 | T. [symbol] | F | 30 | 0 | 0 | 0 |
| 32 | A. L. | M | 65 | 0 | 0 | 0 |
| 33 | A. Y. | F | [symbol] 5 | 0 | 0 | 0 |
| 34 | R. A. | F | 36 | 0 | 0 | 0 |
| 35 | A. [symbol] | M | 27 | 0 | 0 | 0 |
| 36 | B. B. | M | 41 | 0 | 0 | 0 |
| 37 | A. M. | F | 35 | 0 | 0 | 0 |
| 38 | D. N. | M | 35 | 0 | 0 | 0 |
| 39 | [symbol] N. | F | 42 | 0 | 0 | 0 |
| 40 | S. V. | F | 33 | 0 | 0 | 0 |
| 41 | Z. N. | F | 4 [symbol] | 0 | 0 | 0 |
| 42 | D. C. | F | 52 | 0 | 0 | 0 |
| 43 | S. [symbol] | F | 30 | 0 | 0 | 0 |
| 44 | S. M. | F | 63 | 0 | 0 | 0 |
| 45 | M. N. | F | 55 | 0 | 0 | 0 |
| 46 | E. L. | F | 47 | 0 | 0 | 0 |
| 47 | N. L. | F | 34 | 0 | 0 | 0 |
| 48 | H. N. | F | 47 | 0 | 0 | 0 |
| 49 | O. A. | F | [symbol] 2 | 0 | 0 | 0 |
| 50 | M. A. | F | 63 | 0 | 0 | 0 |

[symbol]

indicates data missing or illegible when filed

The original patch sites exhibited no reactions during the induction phase, the rest period or the challenge phase. No other reactions were exhibited.

It is therefore concluded that the test material did not induce a contact dermal irritation and/or sensitization in human subjects in the challenge phase (tenth application).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A composition comprising myrtle berries;
seaweed; and at least two agents selected from the group consisting of tea tree oil, lemongrass oil, evening primrose oil, ginger, jojoba, neem oil, and chamomile; wherein said at least two agents comprise tea tree oil and lemongrass oil; or wherein said at least two agents comprise evening primrose oil, jojoba oil and neem oil; or wherein said composition myrtle berry oil, evening primrose oil, neem oil, jojoba oil and almond oil.

2-4. (canceled)

5. The composition of claim 1, wherein said seaweed is selected from the group consisting of green algae, red algae and brown algae.

6. (canceled)

7. The composition of claim 1, wherein a concentration of said myrtle berries is in the range of from about 10 to about 30% (w/v) of total composition.

8. (canceled)

9. The composition of claim 1, wherein a concentration of said green algae is in the range of from about 5 to about 30% (w/v).

10. The composition of claim 9, wherein a concentration of said green algae is about 10-15% (w/v) of total composition.

11. The composition of claim 1, wherein a concentration of said tea tree oil is in the range of from about 5 to about 20% (v/v) of total composition.

12. The composition of claim 11, wherein a concentration of said tea tree oil is about 12-15% (v/v) of total composition.

13. The composition of claim 1, wherein a concentration of said lemongrass oil is in the range of from about 5 to about 20% (v/v) of total composition.

14. (canceled)

15. The composition of claim 3, wherein a concentration of said evening primrose oil is in the range of from about 5 to about 30% (w/v) of total composition.

16. The composition of claim 15, wherein a concentration of said evening primrose oil is in the range of from about 15 to about 20% (w/v) of total composition.

17. The composition of of claim 3, wherein a concentration of said jojoba oil is in the range of from about 15 to about 30% (w/v) of total composition.

18. The composition of claim 17, wherein a concentration of said jojoba oil is in the range of from about 20 to about 25% (w/v) of total composition.

19. The composition of of claim 3, wherein a concentration of said neem oil is in the range of from about 15 to about 30% (w/v) of total composition.

20. The composition of claim 19, wherein a concentration of said neem oil is in the range of from about 20 to about 25% (w/v) of total composition.

21. The composition of claim 4, wherein a concentration of said almond oil is in the range of from about 15 to about 30% (w/v) of total composition.

22. The composition of claim 21, wherein a concentration of said almond oil is about 20% (w/v) of total composition.

23. The composition of of claim 1 to further comprising an emulsifier, wherein said emulsifier is a surfactant selected from the group consisting of an ionic surfactant, and a non-ionic surfactant.

24-31. (canceled)

32. A method of treating a skin condition selected from the group consisting of psoriasis, eczema, urticaria, scabies, acne, alopecia areata, bullous pemphigoid, dermatitis, impetigo, keloids, pruritis, rosacea, vitiligo, burns, warts, birthmarks, rashes, pigmentation marks, irritation, inflammation, fungal infection, open wounds, dry skin, photodamage and visible signs of aging, the method comprising contacting the skin in the area of the condition with a composition comprising myrtle berries; seaweed; and at least two agents selected from the group consisting of tea tree oil, lemongrass oil, evening primrose oil, ginger, jojoba oil, neem oil, and chamomile.

33. A method of treating a skin condition selected from the group consisting of psoriasis, eczema, urticaria, scabies, acne, alopecia areata, bullous pemphigoid, dermatitis, impetigo, keloids, pruritis, rosacea, vitiligo, burns, warts, birthmarks, rashes, pigmentation marks, irritation, inflammation, fungal infection, open wounds, dry skin, photodamage and visible signs of aging, the method comprising contacting the skin in the area of the condition with a composition comprising myrtle berries, evening primrose oil, neem oil, jojoba oil, and almond oil.

34. (canceled)
